(19) United States
(12) Patent Application Publication
(10) Pub. No.: US 2024/0139191 A1
(43) Pub. Date: May 2, 2024

(54) SMALL MOLECULES AS LARP1 LIGANDS

(71) Applicant: UNIVERSITY OF PITTSBURGH-OF THE COMMONWEALTH SYSTEM OF HIGHER EDUCATION, Pittsburgh, PA (US)

(72) Inventors: Jacob D. DURRANT, Pittsburgh, PA (US); Andrea Jeanne BERMAN, Pittsburgh, PA (US); Kevin Christopher CASSIDY, Pittsburgh, PA (US); Roni Marta LAHR, Pittsburgh, PA (US)

(21) Appl. No.: 18/265,740

(22) PCT Filed: Dec. 14, 2020
(86) PCT No.: PCT/US2021/063043

§ 371 Date: Jun. 7, 2023

Related U.S. Application Data
(60) Provisional application No. 63/125,152, filed on Dec. 14, 2020.

(51) Int. Cl. A61K 31/517; A61K 31/167; A61K 31/415; A61K 31/4155; A61K 31/4412; A61P 31/14

(52) U.S. Cl. CPC A61K 31/517; A61K 31/167; A61K 31/415; A61K 31/4155; A61K 31/4412; A61P 31/14

(57) ABSTRACT

Disclosed are compounds and compositions that bind to the La-related protein 1 (LARP1). LARP1 has roles as both a repressor of translation and stabilizer of mRNA transcripts, and therefore functions as a molecular switch in cancer biology. LARP1 also upregulates the proliferation of RNA viruses. Accordingly, the compounds and compositions disclosed herein that bind to LARP1 may be used for treating or preventing cancer or viral infections in a subject.

### CROSS-REFERENCE TO RELATED APPLICATIONS

This application claims the benefit of priority to U.S. Provisional Application No. 63/125,152 filed Dec. 14, 2020, which is hereby incorporated herein by reference in its entirety.

### BACKGROUND

Cancer is a disease on the rise, from a current worldwide incidence of more than 18 million cases per year to a predicted 40 million cases per year by 2030. Increased cell growth and proliferation and decreased cell attrition are classic hallmarks of cancer. Chemotherapy has remained the mainstay of cancer therapy worldwide since its introduction in the 1940s. Although these drugs have dramatically improved breast cancer, melanoma, and renal-cell-cancer survival, their impact on others such as cancers of the stomach, brain, ovary, and lung has been negligible. Moreover, these treatments are largely non-specific, have toxic side-effects, and are susceptible to resistance. In these and many other cancers, there is a need for new treatment paradigms that target a specific signaling pathway.

Deregulated cell proliferation in cancer exerts a high demand on gene expression that leads to mRNA translation and de novo protein synthesis via assembly of new ribosomes through a process known as ribosome biogenesis. In mammals and other metazoans, the mammalian target of rapamycin-1 (mTORC1) signaling pathway is at the heart of a system that orchestrates ribosome biogenesis and translation of proteins in response to changes in nutrients, energy supplies, or reactive oxygen species. Currently, several mTORC1 inhibitors (mostly rapamycin derivatives) are used as drugs for the treatment of cancer. However, mTORC1 controls many pathways and thus, inhibition of mTORC1 leads to side effects that complicate the long-term therapeutic use of mTORC1 inhibitors. The literature also describes cases of mTORC1-inhibitor resistance. Faes et al., Oxid Med Cell Longev 2017: 1726078. There is a need for new compounds and methods for treating cancer.

The RNA-binding protein La-related protein 1 (LARP1) plays a central role in ribosome biosynthesis. Its C-terminal DM15 region binds the 7-methylguanosine (m⁷G) cap and 5′ terminal oligopyrimidine (TOP) motif characteristic of transcripts encoding ribosomal proteins and translation factors. Under the control of mammalian target of rapamycin complex 1 (mTORC1), LARP1 regulates translation of these transcripts. Additionally, LARP1 depletion has been observed to reduce the levels of some TOP-encoded proteins. Given its potential role as both a repressor of translation and stabilizer of mRNA transcripts, LARP1 functions as a molecular switch in cancer biology. There is a need for small-molecule modulators of the mTORC1-LARP1 axis, thus providing therapeutic potential. Targeting the downstream LARP1 protein can bypass side effects of mTORC1 inhibition by limiting inhibition to the mTORC1-LARP1 axis.

Further, proteomics studies have implicated hundreds of host-cell proteins in SARS-CoV-2 pathogenesis. Among these are proteins required for viral proliferation that are known to interact with the SARS-CoV-2 capsid protein. LARP1 is a particularly promising therapeutic target because it may play a pivotal role in viral replication and propagation. In uninfected human cells, it plays a key role in repressing the expression of the translation apparatus itself. However, in viral-infected cells, LARP1 stimulates viral proliferation. LARP1 is known to upregulate the proliferation of other RNA viruses, including zika, dengue, and influenza. There is a need for compounds that disrupt the interaction between LARP1 and the RNA genome that will inhibit viral proliferation and so may serve as potential therapeutics. LARP1 is known to upregulate the proliferation of other RNA viruses, including zika, dengue, and influenza.

The compounds, compositions, and methods disclosed herein address these and other needs.

### SUMMARY

In accordance with the purposes of the disclosed materials and methods, as embodied and broadly described herein, the disclosed subject matter, in one aspect, relates to compounds, compositions, and methods of making and using compounds and compositions. The compounds disclosed herein may bind the La-related protein 1 (LARP1) and therefore can be used for treating or preventing cancer or viral infections in a subject.

In some aspects, methods of treating or preventing a condition in a subject in need thereof, comprising administering to the subject a therapeutically effective amount of a LARP1-binding compound having a structure according to Formula I or Formula II,

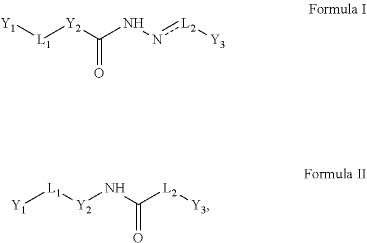

wherein
Y₁ is optionally present, and when present Y₁ is selected from a substituted or unsubstituted aryl, or substituted or unsubstituted heteroaryl;
Y₂ is selected from substituted or unsubstituted aryl, or substituted or unsubstituted heteroaryl;
Y₃ is selected from substituted or unsubstituted aryl, substituted or unsubstituted heteroaryl, substituted or unsubstituted cycloalkyl, substituted or unsubstituted cycloheteroalkyl, substituted or unsubstituted cycloalkenyl, or substituted or unsubstituted cycloheteroalkenyl;
L₁ is selected from a bond, amide, carbonyl, amine, nitro, oxy, hydroxyl, cyano, sulfide, sulfoxide, sulfonyl, sulfonamide, sulfoximine, sulfur diimide, carbamate, thiocarbamate, ester, ether, cyano, halogen, carboxyl, isocyano, C₁-C₃ alkyl, C₁-C₃ haloalkyl, C₁-C₃ haloalkoxy, C₁-C₃ alkoxy, or a combination thereof, wherein L₁ is optionally substituted with halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy;
L₂ is a linker selected from a bond, amide, carbonyl, amine, oxy, sulfide, sulfoxide, sulfonyl, sulfonamide, sulfoximine, sulfur diimide, carbamate, thiocarbamate, ester, ether, carboxyl, isocyano, or a combination thereof, wherein L₂ is optionally substituted with halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy; and
[symbol] represents a bond that is absent or present; or
a pharmaceutically acceptable derivative thereof, are disclosed.

### DETAILED DESCRIPTION

The materials, compounds, compositions, and methods described herein may be understood more readily by reference to the following detailed description of specific aspects of the disclosed subject matter and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an analog” includes mixtures of two or more such analogs, and the like.

“Optional” or “optionally” means that the subsequently described event or circumstance can or cannot occur, and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

The term “binding” or other forms of the word, such as “bind” or “binder,” refer to compounds that target LARP1, and may decrease, inhibit, or enhance the activity or function of LARP1. In some instances, binding of the compounds to LARP1 do not affect the function of LARP1. The compounds disclosed herein could bind LARP1 resulting in a reduction, suppression, increase, and/or enhancement of a given condition, symptom, disorder, or disease, or a significant decrease or increase in the baseline activity of a biological activity or process.

By “reduce” or other forms of the word, such as “reducing” or “reduction,” is meant lowering of an event or characteristic (e.g., tumor growth, metastasis). Similarly, by “increase” or other forms of the word, such as “increasing” or “increase,” we mean the raising of an event or characteristic. It is understood that in both cases this is typically in relation to some standard or expected value, in other words it is relative, but that it is not always necessary for the standard or relative value to be referred to. For example, “reduces tumor growth” means decreasing the amount of tumor cells relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviating of one or more symptoms (such as tumor growth, metastasis, or viral spread), diminishing the extent of cancer or viral infection, stabilizing (i.e., not worsening) state of disease, preventing or delaying spread (e.g., metastasis) of the cancer or viral infection, delaying occurrence or recurrence of disease, delaying or slowing of disease progression, ameliorating the disease state, and remission (whether partial or total).

The term “patient” preferably refers to a human in need of treatment with an anti-cancer or anti-viral agent or treatment for any purpose, and more preferably a human in need of such a treatment to treat cancer, a precancerous condition or lesion, or a viral infection. However, the term “patient” can also refer to non-human animals, preferably mammals such as dogs, cats, horses, cows, pigs, sheep and non-human primates, among others, that are in need of treatment with an anti-cancer or anti-viral agent or treatment.

### Chemical Definitions

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The symbol Aⁿ is used herein as merely a generic substituent in the definitions below.

The term “alkoxy” as used herein is an alkyl group bound through a single, terminal ether linkage; that is, an “alkoxy” group can be defined as —OA¹ where A¹ is alkyl as defined above.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl and heteroaryl group can be substituted or unsubstituted. The aryl and heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” is a short hand notation for C═O.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O⁻.

The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “nitro” as used herein is represented by the formula —NO₂.

The term “cyano” as used herein is represented by the formula —CN

The term “azido” as used herein is represented by the formula —N₃.

The term “sulfonyl” is used herein to refer to the sulfo-oxo group represented by the formula —S(O)₂A¹, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH₂.

The term “thiol” as used herein is represented by the formula —SH.

It is to be understood that the compounds provided herein may contain chiral centers. Such chiral centers may be of either the (R-) or (S-)configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-)form is equivalent, for compounds that undergo epimerization in vivo, to administration of the compound in its (S-)form.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A “pharmaceutically acceptable” component is one that is suitable for use with humans and/or animals without undue adverse side effects (such as toxicity, irritation, and allergic response) commensurate with a reasonable benefit/risk ratio.

“Pharmaceutically acceptable salt” refers to a salt that is pharmaceutically acceptable and has the desired pharmacological properties. Such salts include those that may be formed where acidic protons present in the compounds are capable of reacting with inorganic or organic bases. Suitable inorganic salts include those formed with the alkali metals, e.g., sodium, potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). When two acidic groups are present, a pharmaceutically acceptable salt may be a mono-acid-mono-salt or a di-salt; similarly, where there are more than two acidic groups present, some or all of such groups can be converted into salts.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In reference to viral infections, an effective amount comprises an amount sufficient to cure, palliate, ameliorate, stabilize, reverse, prevent, slow or delay the progression of the disease, pathological condition, or disorder. In some embodiments, an effective amount is an amount sufficient to delay development or infection. In some embodiments, an effective amount is an amount sufficient to prevent or delay occurrence and/or recurrence. An effective amount can be administered in one or more doses. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. In the case of a viral infection, the effective amount of the drug or composition may: cure viral infections, palliate or ameliorate symptoms associated with viral infections, stabilize to some extent and preferably stop viral replication, prevent viral infections or the onset of complications associated with viral infections, slow or delay the progression of viral replication.

Effective amounts of a compound or composition described herein for treating a mammalian subject can include about 0.1 to about 1000 mg/Kg of body weight of the subject/day, such as from about 1 to about 100 mg/Kg/day, especially from about 10 to about 100 mg/Kg/day. The doses can be acute or chronic. A broad range of disclosed composition dosages are believed to be both safe and effective.

Reference will now be made in detail to specific aspects of the disclosed materials, compounds, compositions, articles, and methods, examples of which are illustrated in the accompanying Examples.

### Compounds

Disclosed herein are compounds that bind the La-related protein 1 (LARP1). The compounds can have a structure according to Formula I or Formula II,

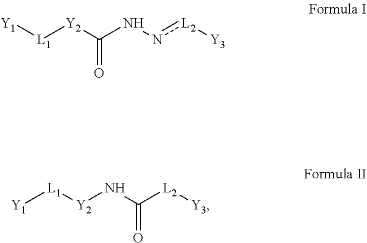

wherein
Y₁ is optionally present, and when present Y₁ is selected from a substituted or unsubstituted aryl, or substituted or unsubstituted heteroaryl;
Y₂ is selected from substituted or unsubstituted aryl, or substituted or unsubstituted heteroaryl;
Y₃ is selected from substituted or unsubstituted aryl, substituted or unsubstituted heteroaryl, substituted or unsubstituted cycloalkyl, substituted or unsubstituted cycloheteroalkyl, substituted or unsubstituted cycloalkenyl, or substituted or unsubstituted cycloheteroalkenyl;
L₁ is selected from a bond, amide, carbonyl, amine, nitro, oxy, hydroxyl, cyano, sulfide, sulfoxide, sulfonyl, sulfonamide, sulfoximine, sulfur diimide, carbamate, thiocarbamate, ester, ether, cyano, halogen, carboxyl, isocyano, C₁-C₃ alkyl, C₁-C₃ haloalkyl, C₁-C₃ haloalkoxy, C₁-C₃ alkoxy, or a combination thereof, wherein L₁ is optionally substituted with halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy;
L₂ is a linker selected from a bond, amide, carbonyl, amine, oxy, sulfide, sulfoxide, sulfonyl, sulfonamide, sulfoximine, sulfur diimide, carbamate, thiocarbamate, ester, ether, carboxyl, isocyano, or a combination thereof, wherein L₂ is optionally substituted with halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy; and
[symbol] represents a bond that is absent or present; or
a pharmaceutically acceptable derivative thereof.

In some aspects of the compounds, Y₂ is selected from substituted or unsubstituted aryl, or substituted or unsubstituted heteroaryl. For example, Y₂ can be selected from substituted or unsubstituted phenyl, or substituted or unsubstituted C₂-C₄ heteroaryl. In specific examples, Y₂ can be selected from phenyl, furyl, imidazolyl, triazolyl, triazinyl, isoxazoyl, thiazolyl, isothiazoyl, pyrazolyl, pyrrolyl, pyrazinyl, tetrazolyl, pyridyl, thienyl, or pyrimidinyl. Y₂ can be substituted or unsubstituted. Exemplary substituents on Y₂ can include, but are not limited to one or more groups selected from alkyl (for e.g., C₁-C₆ alkyl, C₁-C₃ alkyl, or methyl), halogenated alkyl, alkoxy, alkenyl, alkynyl, aryl, heteroaryl, aldehyde, amine, carboxylic acid, ester, ether, halide (for e.g., fluoro, chloro, or bromo), hydroxy, ketone, nitro, silyl, sulfo-oxo, sulfonyl, sulfone, sulfoxide, or thiol. In some instances, Y₂ is selected from a substituted phenyl.

In some aspects of the compounds, Y₃ is selected from substituted or unsubstituted aryl, substituted or unsubstituted heteroaryl, substituted or unsubstituted cycloheteroalkyl, or substituted or unsubstituted cycloheteroalkenyl. For example, Y₃ can be selected from phenyl, triazinyl, oxazoyl, pyridyl, pyridinone, pyrimidinyl, indolyl, isoquinolyl, quinolyl, benzothienyl, benzofuranyl, benzopiperidyl, benzo dihydropyrimidinedione, benzoxazolyl, benzimidazoly, benzothiazolyl, isoindolyl, indolinyl, isoindolinyl, quinoxalinyl, quinazolinyl, cinnolinyl, naphthalyl, [2,3-c] or [3,2-c]-thienopyridyl, tetrahydroquinolyl, dihydroquinolyl, dihydroisoquinolyl, or a combination thereof. Y₃ can be substituted or unsubstituted. Exemplary substituents on Y₃ can include, but are not limited to one or more groups selected from alkyl (for e.g., C₁-C₆ alkyl, C₁-C₃ alkyl, or methyl), halogenated alkyl (for e.g., C₁-C₃ haloalkyl), alkoxy (for e.g., C₁-C₃ alkoxy), alkenyl, alkynyl, aryl, heteroaryl, aldehyde, amine, carboxylic acid, ester, ether, halide (for e.g., fluoro, chloro, or bromo), hydroxy, ketone, nitro, silyl, sulfo-oxo, sulfonyl, sulfone, sulfoxide, thiol, oxo, cyano, carbonyl, carboxyl, amino, isocyano, or C₁-C₃ haloalkoxy.

In some aspects of the compounds, the compounds can have a structure according to Formula I-A or Formula I-B below:

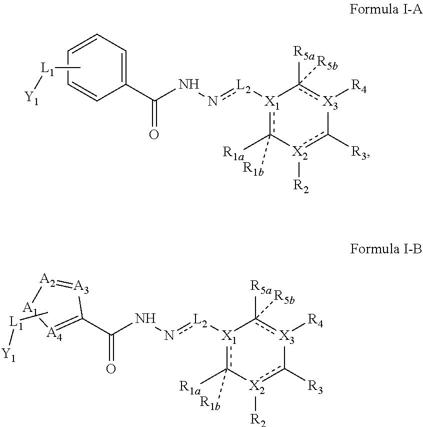

wherein
Y₁ is optionally present, and when present Y₁ is selected from a substituted or unsubstituted aryl, or substituted or unsubstituted heteroaryl;

L₁ is selected from a bond, amide, carbonyl, amine, nitro, oxy, hydroxyl, cyano, sulfide, sulfoxide, sulfonyl, sulfonamide, sulfoximine, sulfur diimide, carbamate, thiocarbamate, ester, ether, cyano, halogen, carboxyl, isocyano, C₁-C₃ alkyl, C₁-C₃ haloalkyl, C₁-C₃ haloalkoxy, C₁-C₃ alkoxy, or a combination thereof, wherein L₁ is optionally substituted with halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy;
L₂ is a linker selected from a bond, amide, carbonyl, amine, oxy, sulfide, sulfoxide, sulfonyl, sulfonamide, sulfoximine, sulfur diimide, carbamate, thiocarbamate, ester, ether, carboxyl, isocyano, or a combination thereof, wherein L₂ is optionally substituted with halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy;
A₁, A₂, A₃, and A₄ are independently selected from C, S, or N;
X₁, X₂, and X₃ are independently selected from C or N;
R₁ₐ and R_{1b} are independently selected from hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₁ₐ and R_{1b} combine to form a carbonyl;
R₂ is selected from hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₁ₐ and R_{1b} combine to form a carbonyl;
R₃ and R₄ are independently selected from hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₃ and R₄ combine with the atoms to which they are attached to form a substituted or unsubstituted aryl, a substituted or unsubstituted heteroaryl, a substituted or unsubstituted cycloalkyl, a substituted or unsubstituted cycloheteroalkyl, a substituted or unsubstituted cycloalkenyl, or a substituted or unsubstituted cycloheteroalkenyl;
R₅ₐ and R_{5b} are independently selected from hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₅ₐ and R_{5b} combine to form a carbonyl; and
[symbol] represents a bond that is absent or present; or
a pharmaceutically acceptable derivative thereof.

In some aspects of the compounds, the compounds can have a structure according to Formula II-A below:

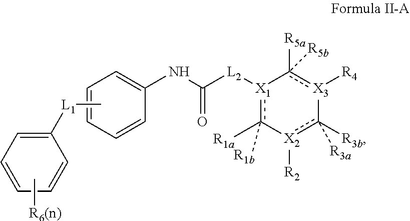

wherein
L₁ is selected from a bond, amide, carbonyl, amine, nitro, oxy, hydroxyl, cyano, sulfide, sulfoxide, sulfonyl, sulfonamide, sulfoximine, sulfur diimide, carbamate, thiocarbamate, ester, ether, cyano, halogen, carboxyl, isocyano, C₁-C₃ alkyl, C₁-C₃ haloalkyl, C₁-C₃ haloalkoxy, C₁-C₃ alkoxy, or a combination thereof, wherein L₁ is optionally substituted with halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy;
L₂ is a linker selected from a bond, amide, carbonyl, amine, oxy, sulfide, sulfoxide, sulfonyl, sulfonamide, sulfoximine, sulfur diimide, carbamate, thiocarbamate, ester, ether, carboxyl, isocyano, or a combination thereof, wherein L₂ is optionally substituted with halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy;
A₁, A₂, A₃, and A₄ are independently selected from C, S, or N;
X₁, X₂, and X₃ are independently selected from C or N;
R₁ₐ and R_{1b} are independently selected from hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₁ₐ and R_{1b} combine to form a carbonyl;
R₂ is selected from hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₁ₐ and R_{1b} combine to form a carbonyl;
R₃ₐ and R_{3b} are independently selected from hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₃ₐ and R_{3b} combine to form a carbonyl;
R₄ are independently selected from hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₃ₐ and R₄ combine with the atoms to which they are attached to form a substituted or unsubstituted aryl, a substituted or unsubstituted heteroaryl, a substituted or unsubstituted cycloalkyl, a substituted or unsubstituted cycloheteroalkyl, a substituted or unsubstituted cycloalkenyl, or a substituted or unsubstituted cycloheteroalkenyl;
R₅ₐ and R_{5b} are independently selected from hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₅ₐ and R_{5b} combine to form a carbonyl; and
R₆ for each occurrence, is independently selected from hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₁ₐ and R_{1b} combine to form a carbonyl;
[symbol] represents a bond that is absent or present;
n is 1, 2, or 3; or
a pharmaceutically acceptable derivative thereof.

In some aspects of the compounds disclosed herein, Y₁ is absent. In other aspects of the compounds, Y₁ is present and selected from selected from substituted aryl, or substituted heteroaryl. For example, Y₁ can be selected from phenyl, triazinyl, oxazoyl, pyridyl, pyridinone, pyrimidinyl, indolyl, isoquinolyl, quinolyl, benzothienyl, benzofuranyl, benzopiperidyl, benzo dihydropyrimidinedione, benzoxazolyl, benzimidazoly, benzothiazolyl, isoindolyl, indolinyl, isoindolinyl, quinoxalinyl, quinazolinyl, cinnolinyl, naphthalyl, [2,3-c] or [3,2-c]-thienopyridyl, tetrahydroquinolyl, dihydroquinolyl, dihydroisoquinolyl, or a combination thereof. Y₁ can be substituted or unsubstituted. Exemplary substituents on Y₁ can include, but are not limited to one or more groups selected from alkyl (for e.g., C₁-C₆ alkyl, C₁-C₃ alkyl, or methyl), halogenated alkyl (for e.g., C₁-C₃ haloalkyl), alkoxy (for e.g., C₁-C₃ alkoxy), alkenyl, alkynyl, aryl, heteroaryl, aldehyde, amine, carboxylic acid, ester, ether, halide (for e.g., fluoro, chloro, or bromo), hydroxy, ketone, nitro, silyl, sulfo-oxo, sulfonyl, sulfone, sulfoxide, thiol, oxo, cyano, carbonyl, carboxyl, amino, isocyano, or C₁-C₃ haloalkoxy.

In some aspects of the compounds disclosed herein, L₁ can be selected from a bond or —R′CO₂R″, —R′CO₂R″—, —R′CONR″R′″, —R′CONR″R′″—, —R′CONHOR″, —R′CONHCN, —R′CHO, —R′NR″R′″, —R′NR″R′″—, —R′NR″COR′″, —R′NR″COR′″—, —R′CONHOH, —R′CONHCN, —R′SO₃H, —R′SOR″—, —R′SO₂R″—, —R′S(O)(NR″)R′″—, —R′SO₂NHCOR″, R′SO₂NHCOR″—, —R′CONHSO₂R″, R′CONHSO₂R″—, —R′SO₂NHR″, or —R′SO₂NR″R′R″—, —R′S(O)NR″R′″, or —R′S(O)NR″R′″—, wherein R′, R″, and R′″ are independently present or absent, when present, R′, R″, and R′″ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkylcycloalkyl, cycloalkenyl, alkylcycloalkenyl, cycloheteroalkyl, alkylcycloheteroalkyl, cycloheteroalkenyl, aryl, alkylaryl, heteroaryl, alkylheteroaryl, or R″ and R′″ together with the atom to which they are attached combine to form a 5-6 membered ring. In some examples, L₁ can be a bond. In some examples, L₁ can be an amide such as —R′CONR″R′″, or —R′CONR″R′″—.

In some aspects of the compounds disclosed herein, Y₁ can be absent and L₁ selected from an amide, carbonyl, amine, nitro, hydroxyl, cyano, sulfide, sulfoxide, sulfonyl, sulfonamide, sulfoximine, sulfur diimide, carbamate, thiocarbamate, ester, ether, cyano, halogen, carboxyl, isocyano, C₁-C₃ alkyl, C₁-C₃ haloalkyl, C₁-C₃ haloalkoxy, C₁-C₃ alkoxy, or a combination thereof, wherein L₁ is optionally substituted with halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy.

In some aspects of the compounds disclosed herein, L₂ can be selected from —R′COR″—, —R′CO₂R″—, —R′CONR″R′″—, —R′NR″R′″—, —R′CONHOH, —R′CONHCN, —R′SOR″—, —R′SO₂R″—, —R′S(O)(NR″)R′″—, R′SO₂NHCOR″—, R′CONHSO₂R″—, —R′SO₂NR″R′″—, or —R′S(O)NR″R′″—, wherein R′, R″, and R′″ are independently present or absent, when present, R′, R″, and R′″ are independently selected from C₁-C₆ alkyl, C₂-C₆ alkenyl, C₂-C₆ alkynyl, C₁-C₆ alkoxy, or C₁-C₆ haloalkyl.

The compounds disclosed herein can have a structure below:

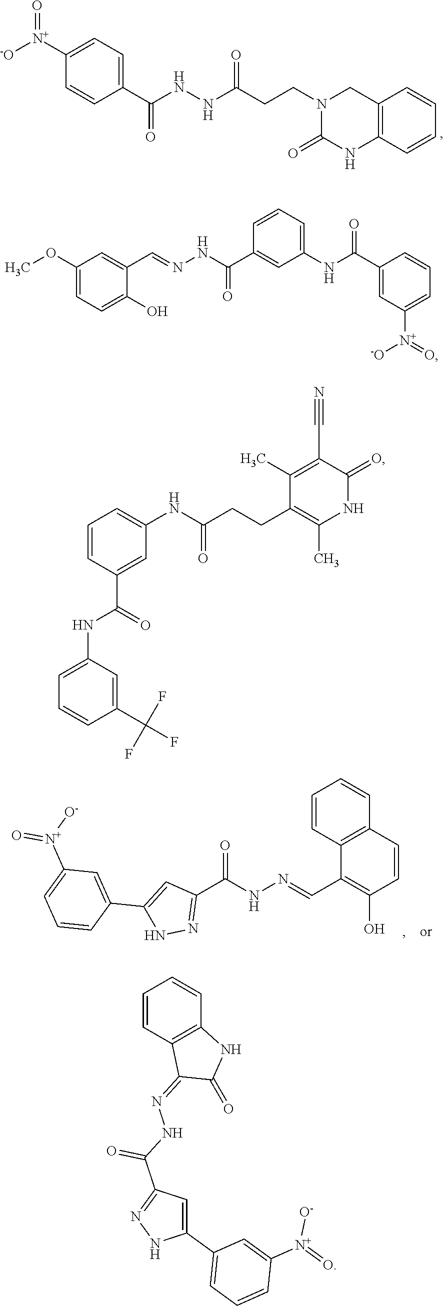

### Methods of Use

The compounds disclosed may bind the La-related protein 1 (LARP1). In some embodiments, binding of LARP1 results in inhibition or suppression of LARP1 activity. In other embodiments, binding of LARP1 results in an upregulation of LARP1 activity. LARP1 is a downstream target of mTORC1 that relays signals from mTORC1 via regulating translation and influencing mRNA stability. Analogous to other downstream targets of mTORC1, LARP1 instigates cell proliferation and is a molecular switch in the onset and maintenance of several forms of cancer. Levels of LARP1 protein correlate with increasing disease progression in cervical cancer where there are stepwise elevations in LARP1 expression through the pre-invasive stages and into invasive disease.

Disclosed herein are methods of treating or preventing cancer in a subject. The disclosed compounds can exert anticancer effects due to binding of LARP1. The method of use can comprise administering to the subject, a therapeutic effective amount of a compound or composition as disclosed herein. The methods can further comprise administering a second compound or composition, such as, for example, anticancer agents or anti-inflammatory agents. Additionally, the method can further comprise administering an effective amount of ionizing radiation to the subject.

The disclosed methods can optionally include identifying a patient who is or can be in need of treatment of cancer. The patient can be a human or other mammal, such as a primate (monkey, chimpanzee, ape, etc.), dog, cat, cow, pig, or horse, or other animals having an oncological disorder. The cancer can be selected from the group consisting of a solid tumor, lung cancer, melanoma, renal-cell cancer, stomach cancer, brain cancer, ovarian cancer, pancreatic cancer, epithelial cancer including non-small cell lung carcinoma, endometrial cancer, breast cancer, hepatocellular cancer, prostate cancer, and cervical cancer, multiple myeloma, mantle cell lymphoma, colorectal cancer, cancer of the anus, bile duct, bladder, bone, bone marrow, bowel (including colon and rectum), eye, gall bladder, kidney, mouth, larynx, esophagus, stomach, testis, cervix, head, neck, ovary, mesothelioma, neuroendocrine, penis, skin, spinal cord, thyroid, vagina, vulva, uterus, liver, muscle, blood cells (including lymphocytes and other immune system cells), brain, carcinomas, Kaposi's sarcoma, melanoma, mesothelioma, soft tissue sarcoma, leukemia (acute lymphoblastic, acute myeloid, chronic lymphocytic, chronic myeloid, and other), and multiple myeloma.

Other examples of cancers that can be treated according to the methods disclosed herein are adrenocortical carcinoma, adrenocortical carcinoma, cerebellar astrocytoma, basal cell carcinoma, Burkitt's lymphoma, carcinoid tumor, central nervous system lymphoma, chronic myeloproliferative disorders, cutaneous T-cell lymphoma, endometrial cancer, ependymoma, esophageal cancer, gastric (stomach) cancer, gastrointestinal carcinoid tumor, germ cell tumor, glioma, hairy cell leukemia, head and neck cancer, hypopharyngeal cancer, hypothalamic and visual pathway glioma, intraocular melanoma, retinoblastoma, islet cell carcinoma (endocrine pancreas), laryngeal cancer, lip and oral cavity cancer, medulloblastoma, Merkel cell carcinoma, squamous neck cancer with occult mycosis fungoides, myelodysplastic syndromes, myelogenous leukemia, nasal cavity and paranasal sinus cancer, nasopharyngeal cancer, neuroblastoma, non-small cell lung cancer, oral cancer, oropharyngeal cancer, osteosarcoma, paranasal sinus and nasal cavity cancer, parathyroid cancer, penile cancer, pheochromocytoma, pine blastoma and supratentorial primitive neuroectodermal tumor, pituitary tumor, plasma cell neoplasm/multiple myeloma, pleuropulmonary blastoma, retinoblastoma, rhabdomyosarcoma, salivary gland cancer, Ewing's sarcoma, soft tissue sarcoma, Sezary syndrome, skin cancer, small cell lung cancer, small intestine cancer, supratentorial primitive neuroectodermal tumors, testicular cancer, thymic carcinoma, thymoma, thyroid cancer, transitional cell cancer of the renal pelvis and ureter, trophoblastic tumor, urethral cancer, uterine cancer, vaginal cancer, vulvar cancer, Waldenström's macroglobulinemia, and Wilms' tumor.

As described herein, the method can further comprise administering an additional therapeutically active co-agent used in the treatment of cancer or anti-cancer therapy. The additional therapeutically active co-agent can be an agent that inhibits the mTOR pathway. For example, the agent can be selected from wortmannin, demethoxyviridin, LY294002, VQD-002, miltefosine, AZD5363, rapamycin, deferolimus, everolimus, temsirolimus, or BEZ235.

In some examples, the anti-cancer therapy can include chemotherapy or radiotherapy. For example, the anti-cancer therapy can include chemotherapy and comprises a chemotherapeutic drug selected from the group consisting of bleomycin, carboplatin, cisplatin, cyclophosphamide, dacarbazine, docetaxel, doxorubicin, etoposide, 5-fluorouracil, folinic acid, gemcitabine, irinotecan, oxaliplatin and paclitaxel, or a combination chemotherapeutic regimen such as AC (doxorubicin and cyclophosphamide), BEP (bleomycin, etoposide and platinum agent), Carbo/taxol (carboplatin and paclitaxel) or FOLFIRINOX (5-fluorouracil, folinic acid, irinotecan, oxaliplatin).

LARP1 is also implicated in the pathogenesis of viruses, such as SARS-CoV2 which causes COVID-19. LARP1 may interact with the SARSCoV2 nucleocapsid protein. Accordingly, disclosed herein are methods of treating or preventing a viral infection in a subject, the method comprising inhibiting La-related protein 1 (LARP1) in the subject in need thereof. In some embodiments, the viral infection can be caused by an RNA virus. For example, the viral infection can include a human coronavirus (e.g., SARS coronavirus or MERS coronavirus), zika, dengue, or influenza.

### Administration

The term “administration” and variants thereof (e.g., “administering” a compound) in reference to a compound disclosed herein means introducing the compound or a prodrug of the compound into the system of the animal in need of treatment. When a compound or prodrug thereof is provided in combination with one or more other active agents, “administration” and its variants are each understood to include concurrent and sequential introduction of the compound or prodrug thereof and other agents.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 2003/0032594 and 2002/0120100 disclose amino acid sequences that can be coupled to another composition and that allows the composition to be translocated across biological membranes. U.S. Application Publication No. 2002/0035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease. For example, the compounds can be used with mutant herpes simplex virus in the treatment of non-small cell lung cancer (Toyoizumi, et al., “Combined therapy with chemotherapeutic agents and herpes simplex virus type IICP34.5 mutant (HSV-1716) in human non-small cell lung cancer,” Human Gene Therapy, 1999, 10(18):17).For the treatment of viral infections, the compounds disclosed herein can be administered to a patient in need of treatment in combination with other antiviral agent. For example, the additional antiviral agent may be a nucleoside reverse transcriptase inhibitors (NRTIs), non-nucleoside reverse transcriptase inhibitors (NNRTIs), protease inhibitors (PIs), integrase inhibitors (INSTIs), fusion inhibitors (FIs), chemokine receptor antagonists, or entry inhibitors. Specific examples of agents include abacavir, acyclovir, acyclovir, adefovir, amantadine, amprenavir, ampligen, arbidol, atazanavir, atripla, balapiravir, BCX4430/Galidesivir, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, favipiravir, fomivirsen, fosamprenavir, foscarnet, fosfonet, ganciclovir, GS-5734/Remdesivir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine zalcitabine, zanamivir, zidovudine, or chloroquine, chloroquine phosphate, hydroxychloroquine, hydroxychloroquine sulfate, Ampligen, APN01, Ganovo, IFX-1, BXT-25, CYNK-001, Tocilizumab, Leronlimab, Ii-key, COVID-19 S-Trimer, Camrelizumab, thymosin, Brilacidin, INO-4800, Prezcobix, cobicistat, mRNA-1273, Arbidol, umifenovir, REGN3048, REGN3051, TNX-1800, fingolimod, methylprednisolone, nitazoxanide, benzopurpin B, C-467929, C-473872, NSC-306711, N-65828, C-21, CGP-42112A, L-163491, xanthoangelol, bevacizumab, polyclonal antibodies derived from patients and monoclonal antibodies (including those antibodies from patients of COVID-19 or monoclonal or polyclonal antibodies which bind SARS-CoV-2), and combinations thereof.

In addition, the compounds of this invention can be combined with compounds that are favorable to preventing lung damage associated with COVID-19, including for example anti-IL-6 and TNF inhibitors, specifically including for example, tocilizumab (Actemra), siltuximab (Sylvant), Tocilizumab, Sarilumab, olokizumab (CDP6038), elsilimomab, BMS-945429 (ALD518), sirukumab (CNTO 136), levilimab (BCD-089), and CPSI-2364 and ALX-0061, ARGX-109, FE301, FM10, infliximab (Remicade), adalimumab (Humira), certolizumab pegol (Cimzia), and golimumab (Simponi), etanercept (Enbrel), Thalidomide (Immunoprin) and its derivatives lenalidomide (Revlimid) and pomalidomide (Pomalyst, Imnovid), xanthine derivatives (e.g. pentoxifylline) and bupropion and 5-HT, agonist hallucinogens including (R)-DOI, TCB-2, LSD and LA-SS-Az.

Therapeutic application of compounds and/or compositions containing them can be accomplished by any suitable therapeutic method and technique presently or prospectively known to those skilled in the art. Further, compounds and compositions disclosed herein have use as starting materials or intermediates for the preparation of other useful compounds and compositions.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina, and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. Nos. 4,608,392; 4,992,478; 4,559,157; and 4,820,508.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

For the treatment of cancer, compounds and agents and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds and agents and compositions disclosed herein can be used in methods of treating cancer wherein the patient is to be treated or is or has been treated with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively. These other substances or radiation treatments can be given at the same as or at different times from the compounds disclosed herein. Examples of other suitable chemotherapeutic agents include, but are not limited to, altretamine, bleomycin, bortezomib (VELCADE), busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib (IRESSA), gemcitabine, hydroxyurea, idarubicin, ifosfamide, imatinib (GLEEVEC), irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uracil, temozolomide, thiotepa, tioguanine/thioguanine, topotecan, treosulfan, vinblastine, vincristine, vindesine, vinorelbine. In an exemplified embodiment, the chemotherapeutic agent is melphalan. Examples of suitable immunotherapeutic agents include, but are not limited to, alemtuzumab, cetuximab (ERBITUX), gemtuzumab, iodine 131 tositumomab, rituximab, trastuzamab (HERCEPTIN). Cytotoxic agents include, for example, radioactive isotopes (e.g., I¹³¹, I¹²⁵, Y⁹⁰, P³², etc.), and toxins of bacterial, fungal, plant, or animal origin (e.g., ricin, botulinum toxin, anthrax toxin, aflatoxin, jellyfish venoms (e.g., box jellyfish), etc.) Also disclosed are methods for treating an oncological disorder comprising administering an effective amount of a compound and/or agent disclosed herein prior to, subsequent to, and/or in combination with administration of a chemotherapeutic agent, an immunotherapeutic agent, a radiotherapeutic agent, or radiotherapy.

For the treatment of a viral infection, compounds and agents and compositions disclosed herein can be administered to a patient in need of treatment prior to, subsequent to, or in combination with other active agents that can treat viral infections.

To provide for the administration of dosages for the desired therapeutic treatment, in some embodiments, pharmaceutical compositions disclosed herein can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: Small Molecules as LARP1 Ligands

This example proposes the use of five small molecule compounds that bind the LARP1 protein, potentially modulating its translational regulatory activity and thus checking the unregulated cellular growth and proliferation usually observed in cancer.

Deregulated cell proliferation in cancer exerts a high demand on gene expression that leads to mRNA translation and de novo protein synthesis via assembly of new ribosomes, organelles that serve as the sites of protein synthesis, through a process known as ribosome biogenesis. In mammals and other metazoans, the mammalian target of rapamycin-1 (mTORC1) signaling pathway is at the heart of a system that orchestrates ribosome biogenesis and translation of proteins in response to changes in nutrients, energy supplies, or reactive oxygen species. Although translation of mRNAs is highly sensitive to the mTORC1 inhibitor rapamycin, there is no evidence of a direct interaction between mTORC1 and the mRNAs that encode components of the translation machinery, such as ribosomal proteins and translation factors. The mechanism by which mTORC1 controls ribosome biogenesis and translation of ribosomal protein transcripts remained elusive until the recent discovery of a novel mTORC1 signaling axis: the mTORC1-LARP1 signaling axis.

La-related protein 1 (LARP1) is a novel downstream target of mTORC1 that relays signals from mTORC1 to other proteins that regulate translation and influence mRNA stability. The precise biological action of LARP1 is still debated. A number of studies suggest that, analogous to other downstream targets of mTORC1, LARP1 instigates cell proliferation and is a key molecular switch in the onset and maintenance of several forms of cancer [Rhodes D R et al, Neoplasia. 2004; 6:1-6]. Levels of LARP1 protein correlate with increasing disease progression in cervical cancer where there are stepwise elevations in LARP1 expression through the pre-invasive stages and into invasive disease [Burrows C et al, Nucleic Acids Res. 2010; 38: 5542-5553]. In hepatocellular cancer (HCC), high levels of LARP1 protein in tumor tissue correlate with tumor size and survival time, leading to an approximately 35% increased risk of death within five years (compared to low levels) [Xie C et al, J. Transl. Med. 2013]. Other studies have demonstrated that LARP1 is associated with the proliferation of endometrial cancer and lung cancer [Tcherkezian J, et al, Genes Dev. 2014; 28 (4):357-371].

Recent work has also implicated LARP1 in the pathogenesis of SARS-CoV2, which causes COVID-19. Others recently found that LARP1 interacts with the SARSCoV2 nucleocapsid protein [Gordon, et al. 2020, bioRxiv preprint]. The mTORC1 inhibitor rapamycin in fact decreases MERS infections (caused by another coronavirus) by 60% [Kindrachuk, J. et al., 2015, Gordon et al., 2020]. Rapamycin may serve as a particularly useful COVID-19 therapeutic in the short term, given that it is already FDA approved. But waves of COVID-19 infection are likely, and rapamycin has problematic side effects. There is again a need for small molecules that can more specifically target the mTORC1-LARP1 axis.

Given the pivotal influence of the mTORC1-LARP1 signaling axis in metabolism, cellular proliferation, cell-death pathways, and viral pathogenesis, small-molecule modulators that may alter the activity of this signaling axis have been developed and thus have therapeutic potential. Although the central regulator mTORC1 might seem to be the more direct drug target, mTORC1 inhibition affects many critical pathways, leading to side effects that complicate the long-term therapeutic use of mTORC I inhibitors.

Exemplified are five small-molecule compounds (Scheme 1) that are expected to bind the LARP1 protein, altering its activity and thus preventing the unregulated cell growth typical of cancer. These small molecules were specifically designed to target the LARP1 RNA-binding DM15 region. The RNA binding function of this domain has been shown to be critical for the efficient production of ribosomal components.

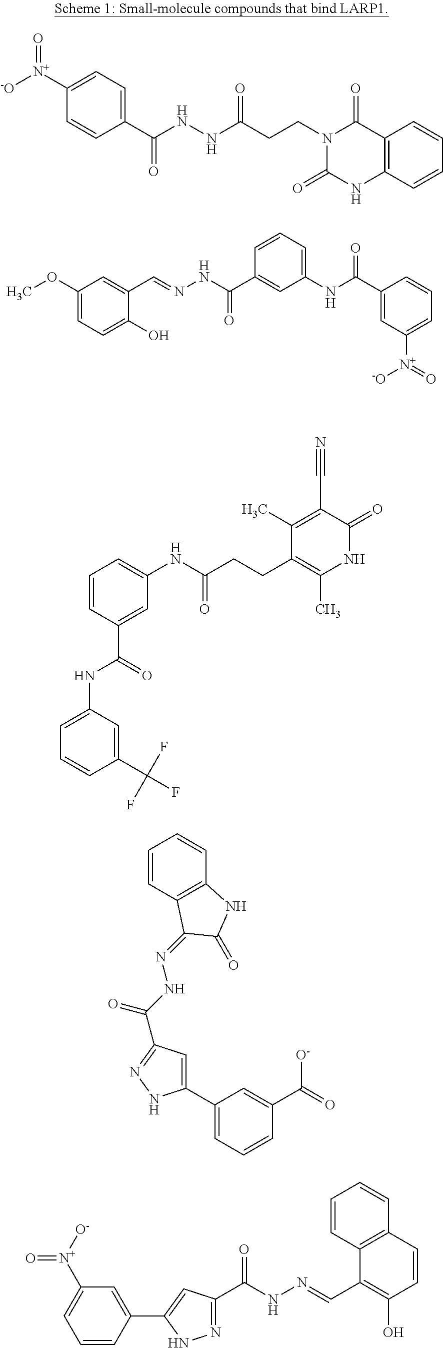

Molecular dynamics simulations have also been performed to investigate the dynamics of the LARP1 DM15 region. The simulations and subsequent analyses provided evidence that pockets on the protein surface are druggable. Based on this discovery, “druggability” simulations were used to identify pharmacophores, or chemical blueprints, that describe the chemical features a molecule preferably has to bind these pockets. These pharmacophore models were then used to identify matching compounds from the pharmit database. Compounds with poor predicted solubility, molecular size, commercial availability, were removed. The remaining compounds were docked into the appropriate LARP1 DM15 pockets. Twenty “top hit” compounds were selected for further in vitro experiments.

Per thermal shift assays, five of these compounds caused an increase in the thermal stability of Human LARP1 DM15 over a DMSO control, suggesting that they bind to the protein. Three of these compounds were shown to cause a greater increase in thermal stability than the naturally occurring ligand m7GpppC (Cap-C). Future efforts to characterize compound binding may include X-ray crystallography, thermal shift assays, and nano isothermal titration calorimetry. Also, determination of the EC₅₀ values (i.e., the compound concentration required to alter the RNA-binding ability of LARP1 DM15 by 50%) via nano isothermal titration calorimetry can be made.

### LARP1 and COVID-19 Application

COVID-19 is caused by severe acute respiratory syndrome coronavirus 2 (SARS-CoV-2), a single-stranded, positive-sense virus of the Coronaviridae family While much is known about the virology of this family, there are no effective treatments or vaccines for SARS-CoV-2 or any human-pathogen Coronaviridae (e.g., SARS, MERS). Proteomics studies have implicated hundreds of host-cell proteins in SARS-CoV-2 pathogenesis. Among these are proteins required for viral proliferation that are known to interact with the SARS-CoV-2 capsid protein. The La-related protein 1 (LARP1) is a particularly promising therapeutic target because it may play a pivotal role in viral replication and propagation. LARP1 is a multidomain RNA-binding protein that is known to upregulate the proliferation of other RNA viruses, including zika, dengue, and influenza. In uninfected human cells, it plays a key role in repressing the expression of the translation apparatus itself. However, in viral-infected cells, LARP1 stimulates viral proliferation. Given the data collected, it is believed that LARP1 interacts with the capsid indirectly, through mutual interactions with the viral RNA genome via LARP1's bona fide 7-methylguanosine cap-binding domain, denoted the DM15 region. It has been demonstrated that the LARP1 DM15 region has a low-nanomolar affinity for the invariant capped nucleotide found at the 5′ end of the SARS-CoV-2 genome. Provided herein are compounds that may disrupt the interaction between LARP1 and the SARS-CoV-2 genome thereby inhibiting viral proliferation, and so serve as potential therapeutics.

Molecular dynamics simulations and in silico screening were applied to a crystal structure of the LARP1 DM15 region. These experiments revealed the underlying movements that may govern RNA binding, identified druggable pockets on the protein surface, and allowed computational testing of these pockets for predicted small-molecule binding. These computational models were used to identify several candidate small-molecule ligands that were subsequently validated experimentally.

It has been discovered that the LARP1 DM15 region is a bona fide RNA-cap-binding module that interacts with human mRNA transcripts encoding the translation machinery. These transcripts are characterized at the 5′ end by the invariant 7-methylguanosine (m⁷G)pppC motif (FIG. 1A). Evidence suggests that LARP1 also plays an important role in the pathogenesis of several single-stranded RNA viruses, including dengue, hepatitis C, zika, and influenza. It was discovered that the DM15 region forms an even tighter interaction with the m⁷GpppA motif characteristic of many ssRNA viruses (e.g., SARS-CoV-2) than with the endogenous (human) m⁷GpppC-initiated mRNA targets (FIGS. 1B & C). The role of LARP1 in the replication of the dengue flavivirus is also being investigated.

Most importantly, LARP1 has been found to interact with the SARS-CoV-2 nucleocapsid (N). As both are RNA-binding proteins, and in light of the direct interactions LARP1 makes with the 5′ cap of dengue virus, we predict that LARP1 and N interact indirectly via mutual binding to the viral genome. Small molecules that prevent this interaction by impeding LARP1/RNA binding could interfere with SARS-CoV-2 replication or viral hijacking of cellular machinery, thereby serving as effective host-directed anti-viral therapies. Given the discovery of novel LARP1 ligands, further identification of compounds that disrupt LARP1/RNA binding is described herein.

Overview: LARP1 has been shown to be an anti-SARS-CoV-2 drug target in part because it is a stable human protein. LARP1 directly binds viral ssRNA and so is believed to play a role in the pathogenesis of many viruses, including multiple coronavirus strains. Using small molecules to modulate LARP1 activity is a novel approach. No drugs or other exogenous compounds are known to bind LARP1 beyond those disclosed herein.

Molecular dynamics simulations and virtual screening provide candidate ligands. To assess whether the LARP1-DM15 region is well suited to in silico structure-based drug discovery (SBDD), a 500 ns molecular-dynamics (MD) simulation was performed that started from 4ZC4 structure, chain B (4ZC4:B). To identify representative conformations from among the many sampled during the simulation, affinity propagation clustering was used to construct a conformational ensemble comprised of 12 distinct DM15 conformations (FIG. 1A). To this ensemble, the 4ZC4:B crystal structure was added as well as the simulated DM15 conformation with the largest cap-binding-pocket volume (LARP1_{large}) because open pockets are often amenable to SBDD.

To develop pharmacophores (i.e., chemical templates) that describe ligand binding to the DM15 cap-binding pocket, DruGUI was used to perform druggability simulations. These simulations capture protein motions in an aqueous solution containing multiple chemical probes, such that the probes are free to diffuse to druggable hotspots on the DM15 surface. Three independent druggability simulations were started from each of the 14 members of the MD-extracted conformational ensemble (42 simulations total). The hotspots were networked together to create pharmacophore models and the pharmit database was searched for matching compounds.

As a second assessment of ligand binding, matching compounds were docked onto the LARP1 surface using AutoDock Vina, which predicts the 3D geometry (i.e., binding pose) of a candidate ligand and evaluates that pose for predicted binding affinity. From these results, twenty predicted ligands with excellent docking scores and reasonable poses were identified. These models and predicted ligands further support that the LARP1-DM15 region can bind small molecules.

Experimental evidence confirms binding. To provide experimental evidence that the LARP1-DM15 region is druggable, the twenty top-scoring compounds were purchased from the virtual screen for testing. In this assay, the hydrophobic fluorescent dye SYPRO Orange is used to monitor the melting curve of protein in the presence and absence of ligand(s). A ligand that stabilizes the protein fold—likely via physical interactions (binding)—increases the melting temperature (Tₘ) of the protein. Although TSA provides only an indirect measurement of binding, it is an excellent tool for first-pass, high-throughput ligand identification that can guide further experimental studies. Five of the tested compounds caused an increase in LARP1 thermal stability, suggesting they do bind the protein. Three caused a greater increase in thermal stability than the known endogenous ligand m⁷GpppC. Subsequent crystallography work and isothermal titration calorimetry (ITC) can further verify binding.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

1. A method of treating or preventing a condition in a subject in need thereof, comprising administering to the subject a therapeutically effective amount of a La-related protein 1 (LARP1) binding compound having a structure according to Formula I or Formula II, 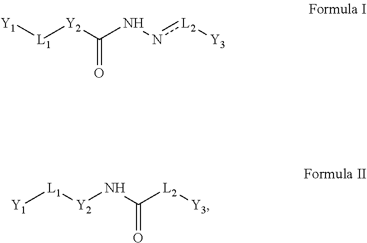
wherein
Y₁ is optionally present, and when present Y₁ is a substituted or unsubstituted aryl, or substituted or unsubstituted heteroaryl;
Y₂ is substituted or unsubstituted aryl, or substituted or unsubstituted heteroaryl;
Y₃ is substituted or unsubstituted aryl, substituted or unsubstituted heteroaryl, substituted or unsubstituted cycloalkyl, substituted or unsubstituted cycloheteroalkyl, substituted or unsubstituted cycloalkenyl, or substituted or unsubstituted cycloheteroalkenyl;
L₁ is a bond, amide, carbonyl, amine, nitro, oxy, hydroxyl, cyano, sulfide, sulfoxide, sulfonyl, sulfonamide, sulfoximine, sulfur diimide, carbamate, thiocarbamate, ester, ether, cyano, halogen, carboxyl, isocyano, C₁-C₃ alkyl, C₁-C₃ haloalkyl, C₁-C₃ haloalkoxy, C₁-C₃ alkoxy, or a combination thereof, wherein L₁ is optionally substituted with halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy;
L₂ is a linker and the linker is a bond, amide, carbonyl, amine, oxy, sulfide, sulfoxide, sulfonyl, sulfonamide, sulfoximine, sulfur diimide, carbamate, thiocarbamate, ester, ether, carboxyl, isocyano, or a combination thereof, wherein L₂ is optionally substituted with halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy; and [symbol]
represents a bond that is absent or present; or
a pharmaceutically acceptable derivative thereof.

2. The method of claim 1, wherein Y₂ is substituted or unsubstituted aryl, or substituted or unsubstituted heteroaryl.

3. The method of claim 1, wherein Y₂ is substituted or unsubstituted phenyl, or substituted or unsubstituted C₂-C₄ heteroaryl.

4. The method of claim 1, wherein Y₂ is phenyl, furyl, imidazolyl, triazolyl, triazinyl, isoxazoyl, thiazolyl, isothiazoyl, pyrazolyl, pyrrolyl, pyrazinyl, tetrazolyl, pyridyl, thienyl, or pyrimidinyl, wherein Y₂ is optionally substituted.

5. The method of claim 1, wherein Y₂ is a substituted phenyl.

6. The method of claim 1, wherein Y₃ is substituted or unsubstituted aryl, substituted or unsubstituted heteroaryl, substituted or unsubstituted cycloheteroalkyl, or substituted or unsubstituted cycloheteroalkenyl.

7. The method of claim 1, wherein Y₃ is phenyl, triazinyl, oxazoyl, pyridyl, pyridinone, pyrimidinyl, indolyl, isoquinolyl, quinolyl, benzothienyl, benzofuranyl, benzopiperidyl, benzo dihydropyrimidinedione, benzoxazolyl, benzimidazoly, benzothiazolyl, isoindolyl, indolinyl, isoindolinyl, quinoxalinyl, quinazolinyl, cinnolinyl, naphthalyl, [2,3-c] or [3,2-c]-thienopyridyl, tetrahydroquinolyl, dihydroquinolyl, dihydroisoquinolyl, or a combination thereof.

8. The method of claim 1, wherein Y₃ is substituted with a moiety, and the moiety is oxo, nitro, cyano, carbonyl, hydroxyl, halogen, carboxyl, amino, isocyano, C₁-C₃ alkyl, C₁-C₃ haloalkyl, C₁-C₃ haloalkoxy, or C₁-C₃ alkoxy.

9. The method according to claim 1, wherein the LARP1 binding compound has a structure according to Formula I-A or Formula I-B, 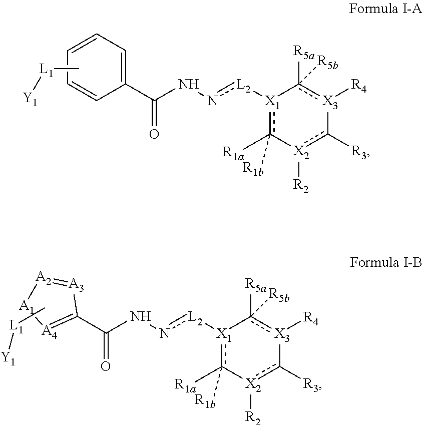
wherein
Y₁ is optionally present, and when present Y₁ is a substituted or unsubstituted aryl, or substituted or unsubstituted heteroaryl;
L₁ is a bond, amide, carbonyl, amine, nitro, oxy, hydroxyl, cyano, sulfide, sulfoxide, sulfonyl, sulfonamide, sulfoximine, sulfur diimide, carbamate, thiocarbamate, ester, ether, cyano, halogen, carboxyl, isocyano, C₁-C₃ alkyl, C₁-C₃ haloalkyl, C₁-C₃ haloalkoxy, C₁-C₃ alkoxy, or a combination thereof, wherein L₁ is optionally substituted with halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy;
L₂ is a linker and the linker is a bond, amide, carbonyl, amine, oxy, sulfide, sulfoxide, sulfonyl, sulfonamide, sulfoximine, sulfur diimide, carbamate, thiocarbamate, ester, ether, carboxyl, isocyano, or a combination thereof, wherein L₂ is optionally substituted with halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy;
A₁, A₂, A₃, and A₄ are independently C, S, or N;
X₁, X₂, and X₃ are independently C or N;
R₁ₐ and R_{1b} are independently hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₁ₐ and R_{1b} combine to form a carbonyl;
R₂ is hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₁ₐ and R_{1b} combine to form a carbonyl;
R₃ and R₄ are independently hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₃ and R₄ combine with the atoms to which they are attached to form a substituted or unsubstituted aryl, a substituted or unsubstituted heteroaryl, a substituted or unsubstituted cycloalkyl, a substituted or unsubstituted cycloheteroalkyl, a substituted or unsubstituted cycloalkenyl, or a substituted or unsubstituted cycloheteroalkenyl;
R₅ₐ and R_{5b} are independently hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₅ₐ and R_{5b} combine to form a carbonyl; and [symbol]
represents a bond that is absent or present; or
a pharmaceutically acceptable derivative thereof.

10. The method according to claim 1, wherein the LARP1 binding compound has a structure according to Formula II-A, 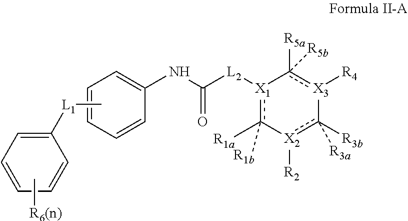
wherein
L₁ is a bond, amide, carbonyl, amine, nitro, oxy, hydroxyl, cyano, sulfide, sulfoxide, sulfonyl, sulfonamide, sulfoximine, sulfur diimide, carbamate, thiocarbamate, ester, ether, cyano, halogen, carboxyl, isocyano, C₁-C₃ alkyl, C₁-C₃ haloalkyl, C₁-C₃ haloalkoxy, C₁-C₃ alkoxy, or a combination thereof, wherein L₁ is optionally substituted with halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy;
L₂ is a linker and the linker is a bond, amide, carbonyl, amine, oxy, sulfide, sulfoxide, sulfonyl, sulfonamide, sulfoximine, sulfur diimide, carbamate, thiocarbamate, ester, ether, carboxyl, isocyano, or a combination thereof, wherein L₂ is optionally substituted with halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy;
A₁, A₂, A₃, and A₄ are independently C, S, or N;
X₁, X₂, and X₃ are independently C or N;
R₁ₐ and R_{1b} are independently hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₁ₐ and R_{1b} combine to form a carbonyl;
R₂ is hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₁ₐ and R_{1b} combine to form a carbonyl;
R₃ₐ and R_{3b} are independently hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₃ₐ and R_{3b} combine to form a carbonyl;
R₄ are independently hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₃ₐ and R₄ combine with the atoms to which they are attached to form a substituted or unsubstituted aryl, a substituted or unsubstituted heteroaryl, a substituted or unsubstituted cycloalkyl, a substituted or unsubstituted cycloheteroalkyl, a substituted or unsubstituted cycloalkenyl, or a substituted or unsubstituted cycloheteroalkenyl;
R₅ₐ and R_{5b} are independently hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₅ₐ and R_{5b} combine to form a carbonyl; and
R₆ for each occurrence, is independently hydrogen, halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy, or R₁ₐ and R_{1b} combine to form a carbonyl; [symbol]
represents a bond that is absent or present;
n is 1, 2, or 3; or
a pharmaceutically acceptable derivative thereof.

11. The method of claim 1, wherein Y₁ is absent.

12. The method of claim 1, wherein Y₁ is substituted aryl, or substituted heteroaryl.

13. The method of claim 12, wherein Y₁ is substituted with a moiety and the moiety is nitro, cyano, hydroxyl, halogen, hydroxyl, carboxyl, amino, isocyano, C₁-C₃ alkyl, C₁-C₃ haloalkyl, C₁-C₃ haloalkoxy, or C₁-C₃ alkoxy.

14. The method of claim 1, wherein L₁ is a bond or —R′CO₂R″, —R′CO₂R″—, —R′CONR″R′″, —R′CONR″R′″—, —R′CONHOR″, —R′CONHCN, —R′CHO, —R′NR″R′″, —R′NR″R′″—, —R′NR″COR′″, —R′NR″COR′″—, —R′CONHOH, —R′CONHCN, —R′SO₃H, —R′SOR″—, —R′SO₂R″—, —R′S(O)(NR″)R′″—, —R′SO₂NHCOR″, R′SO₂NHCOR″—, —R′CONHSO₂R″, R′CONHSO₂R″—, —R′SO₂NHR″, or —R′SO₂NR″R′″—, —R′S(O)NR″R′″, or —R′S(O)NR″R′″—, wherein R′, R″, and R′″ are independently present or absent, when present, R′, R″, and R′″ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkylcycloalkyl, cycloalkenyl, alkylcycloalkenyl, cycloheteroalkyl, alkylcycloheteroalkyl, cycloheteroalkenyl, aryl, alkylaryl, heteroaryl, alkylheteroaryl, or R″ and R′″ together with the atom to which they are attached combine to form a 5-6 membered ring.

15. The method of claim 1, wherein Y₁ is absent and L₁ is amide, carbonyl, amine, nitro, hydroxyl, cyano, sulfide, sulfoxide, sulfonyl, sulfonamide, sulfoximine, sulfur diimide, carbamate, thiocarbamate, ester, ether, cyano, halogen, carboxyl, isocyano, C₁-C₃ alkyl, C₁-C₃ haloalkyl, C₁-C₃ haloalkoxy, C₁-C₃ alkoxy, or a combination thereof, wherein L₁ is optionally substituted with halogen, hydroxyl, amine, cyano, nitro, C₁-C₃ alkyl, C₂-C₄ alkenyl, C₁-C₃ alkoxy, C₁-C₃ haloalkyl, or C₁-C₃ haloalkoxy.

16. The method of claim 1, wherein L₂ is —R′COR″—, —R′CO₂R″—, —R′CONR″R′″—, —R′NR″R′″—, —R′CONHOH, —R′CONHCN, —R′SOR″—, —R′SO₂R″—, —R′S(O)(NR″)R′″—, R′SO₂NHCOR″—, R′CONHSO₂R″—, —R′SO₂NR″R′″—, or —R′S(O)NR″R′″—, wherein R′, R″, and R′″ are independently present or absent, when present, R′, R″, and R′″ are independently selected from C₁-C₆ alkyl, C₂-C₆ alkenyl, C₂-C₆ alkynyl, C₁-C₆ alkoxy, or C₁-C₆ haloalkyl.

17. The method of claim 1, wherein the LARP1 binding compound has a structure below:

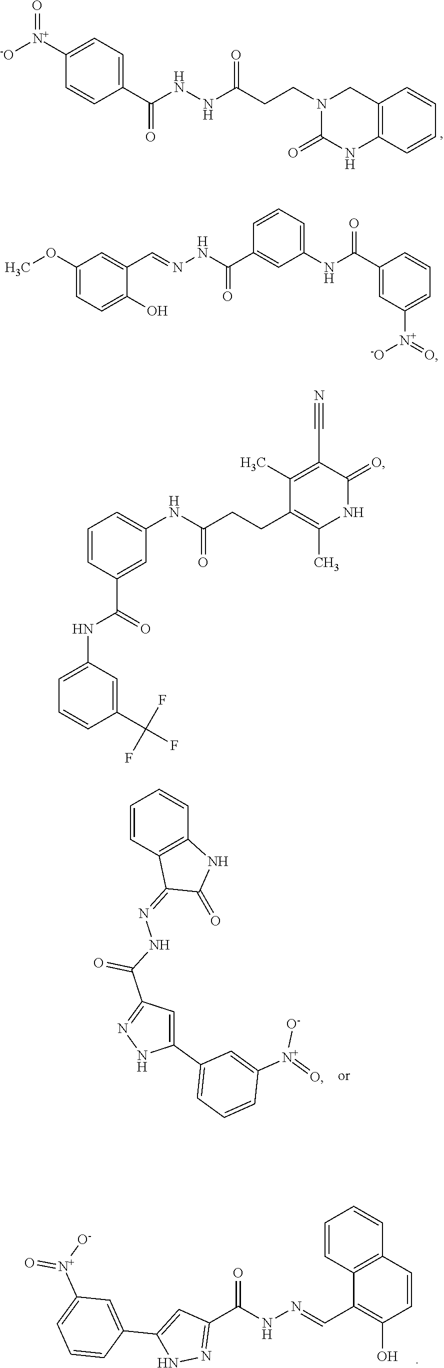

18. The method of claim 1, wherein the condition is cancer or a viral infection.

19. (canceled)

20. (canceled)

21. (canceled)

22. The method of claim 1, further comprising administering an additional therapeutically active co-agent.

23. The method of claim 22, wherein the additional therapeutically active co-agent is an agent that inhibits the mTOR pathway.

24. (canceled)

25. (canceled)

26. (canceled)

27. (canceled)

28. (canceled)

29. (canceled)

30. (canceled)

31. (canceled)
